(19) United States
(12) Patent Application Publication
(10) Pub. No.: US 2009/0264849 A1
(43) Pub. Date: Oct. 22, 2009

(54) DIAPERS, DIAPER WIPES AND DIAPAER PADS WITH ACTIVE INGREDIENTS INTEGRATED THEREIN

(76) Inventor: Timothy Matthew La Croix, San Diego, CA (US)

Correspondence Address: Margaret Anne;APT. 3207, 3535 Lebon Drive, San Diego, CA, 92122, US

(21) Appl. No.: 12/106,279

(22) Filed: Apr. 19, 2008

(51) Int. Cl. A61F 13/15; A47K 7/02

(52) U.S. Cl. 604/385.06; 604/385.01; 604/358; 15/209.1

(57) ABSTRACT

The present invention provides a diaper, diaper pad insert and/or a diaper wipe with active ingredients integrated therein, including ointments for treating diaper rash, baby oils and lotions for treating dry skin, and/or powder treatments for absorbing moisture, including baby powder, corn starch powders and the like. In a first embodiment, the invention provides a diaper having a active ingredients integrated therein comprising a skin-contact surface appointed for contact with an infant's bottom, at least one inner absorption layer, an outer most layer, and a active ingredients segment. The active ingredient segment includes active ingredients associated therewith and is adapted for contact and direct delivery to the infant's bottom when the diaper is placed on and worn by the infant. The active ingredient may be applied as a coating or integrate as fine particles within the skin-contact surface. As the infant wears the diaper and moves around, or the active ingredient wipe is wiped on the infant, the skin-contact surface comes into contact with the infant's body, transferring active ingredients associated with the active ingredient segment onto the infant's body.

### BACKGROUND OF THE INVENTION

1. Field of the Invention

The present invention relates to a diaper, a diaper wipe and/or diaper pads appointed to be inserted into a diaper that include active ingredients integrated therein; and more particularly to a diaper and/or diaper wipe and/or diaper pad with active ingredients integrated therein including diaper rash ointment for treating diaper rash, baby oils and lotions for treating dry skin, and/or powder treatments for absorbing moisture, including baby powder, corn starch powders and the like.

2. Description of the Prior Art

Diaper rash affects virtually all babies at one time or another. The rash ranges from mild outbreaks with few red bumps, while still uncomfortable for the infant, or can be sever with bleeding sores, causing severe pain and discomfort for the infant and worry to the parent or caretakers. Particular problems can arise during overnight wearing of the diaper, as the infant urinates frequently, saturating the diaper with uric acid, and changing of the diaper generally occurs in the morning when the infant/toddler awakens. Even still, where a diaper soiled by an infant's bowl movement is not immediately changed, due to circumstances such as diarrhea, an overnight soiling, traveling or the parent is unaware of the soiling, the infant/toddler's bottom is extremely vulnerable to a diaper rash outbreak.

Despite the problems associated with diaper rash and general maintenance of an infant's bottom, the only recourse is to topically treat the infant's bottom with lathering of the lotion, ointment, and oil or baby powder on the bottom while diapering the infant. Powders and creams or lotions are frequently applied to an infant's bottom to ensure that the infant's skin remains healthy. A plethora of compositions for topical treatments have been provided wherein lotion, ointment or baby powder are applied to the bottom of the infant while diapering, before securing the diaper.

However, such topical application during diapering requires the caregiver/parent to have to fumble with ointment containers/tubes when diapering an infant, which can be quite a challenge when the infant is very active and not wanting to lay still on a changing table or surface. During travel, such ointments are just one more item that needs to be toted around in the diaper bag and dealing with same when using a restroom changing table can be quite difficult. In fact, the situation can be dangerous, as the caregiver must take both hands off of the infant to open the ointment container, leading to the risk of the infant rolling from the changing surface. Moreover, the topical application results in residual ointment or lotion being deposited on the caregiver's fingers. In a perfect situation the caregiver would be able to immediately wash his/her hands. But in reality, the caregiver does not always have that luxury, as the infant cannot be left on the changing table alone while the caregiver goes to wash his/her hands, and it is very difficult washing one's hands while holding an infant. During travel, a sink may not even be readily available, as parents often must change the infant's/baby's diaper in a vehicle or stroller. Realistically, the ointment cannot be immediately washed from the hands after every application, and is transferred onto clothing, or worse, onto bottles and nipples being prepared for feeding the infant, or onto the breast, ultimately risking ingesting by the infant.

Notwithstanding the efforts of prior art workers, there remains a need in the art for a diaper wherein an oil, powder and or ointment is integrated therein so that direct application is not necessary and the caregiver or parent merely attaches the diaper on the infant and direct delivery of the ointment, oil or powder to the infant's bottom results as the diaper is worn. In addition, there exists an art recognized need for a diaper insertion pad that includes an ointment, oil or powder integrated therein that can readily be placed in a diaper to provide protection to the infant's bottom and deliver active ingredients thereto. Further, there is a need in the art for a baby wipe wherein an active ingredients or lotion is integrated therein, so that the wiper can directly apply active ingredients to an infant's bottom as it is being utilized to wipe the infant's bottom. These applications would fill a need in the art by providing protection to the infant's bottom as the diaper is worn, while further providing the parent or caretaker with the ability to diaper an infant with minimum effort and with minimum items to carry.

### SUMMARY OF THE INVENTION

The present invention provides a diaper, diaper pad insert and/or a diaper wipe with active ingredients integrated therein, including ointments for treating diaper rash, baby oils and lotions for treating dry skin, and/or powder treatments for absorbing moisture, including baby powder, corn starch powders and the like. Direct application of the diaper rash ointment, baby oil, lotion and/or powder is achieved while the diaper is being donned and worn as the active ingredient segment is wiped against the infant's body the active ingredients are transferred onto the infant's skin.

In the embodiments concerning the diaper and diaper pad, during wear, the diaper (and/inserted diaper pad) with active ingredients integrated therein comes into contact with the infant's bottom and transfers the active ingredients thereto. Through long intervals, the infant is protected and the risk of developing (or worsening) diaper rash conditions is mitigated. The diaper should optimally be changed as soon as possible after each soiling as a usual diaper, but the diaper (and/inserted diaper pad) herein facilitates in protecting the infant's body during wear and provides advantages during the diapering process.

Advantageous include: (i) protecting an infant from diaper rash during prolonged periods where the infant is not changed—such as during the night, extended periods of sleep, naptime, or during travel; (ii) convenience during the diapering process, allowing the caregiver to simply place the diaper on the child without the need to apply separate ointments, lotions, and I or powder, which can be very advantageous when dealing with a squirming toddler; (iii) provides the caregiver the ability to apply the ointment, or other medicament, without using their hands so that their hands do not have residual ointment or lotion on them; and (iv) results in a lighter, more compact diaper bag, as less items are necessary to carry along in the bag, so that the caregiver will not have to tote around lotions, ointments and powders in the bag, instead the caregiver will need to only carry the diaper and/or diaper wipes with active ingredients integrated therein.

The caregiver could carry different active ingredient diapers (and/or diaper pads), for example, carrying two or three with ointment, and two or three with powder within a single diaper bag. The invention herein is not appointed to replace the frequency of diaper changing, for the diaper should optimally be changed as soon as possible after being soiled to best prevent diaper rash or irritation. However, the diaper herein will provide the ability to diaper an infant in a fast convenient manner, wherein the caregiver simply places the diaper on the infant, without the step of separately applying ointment. This can be very advantageous when dealing with a squirming infant or toddler. Moreover, the caregiver does not have to deal with diaper rash ointment or the like on the hands, which can be difficult to immediately wash if in a location lacking a sink or if dealing with an infant on a changing table, which cannot be left to attend to hand washing. Further, if the diaper is not immediately changed, the ointment transfer from the diaper will facilitate in preventing the rash and mitigate the severity of a potential rash.

In a first embodiment, the invention provides a diaper having a active ingredients integrated therein comprising a skin-contact surface appointed for contact with an infant's bottom, at least one inner absorption layer, an outer most layer, and a active ingredients segment. The active ingredient segment includes active ingredients associated therewith and is adapted for contact and direct delivery to the infant's bottom when the diaper is placed on and worn by the infant. The active ingredient segment is located in association with the skin-contact surface of the diaper so that the active ingredients coated upon or integrated within the skin-contact surface comes into contact with the infant's bottom. As the infant wears the diaper and moves around, the skin-contact surface comes into contact with the infant's body, transferring active ingredients associated with the active ingredient segment onto the infant's body. This active ingredient may be a lotion, baby oil or petroleum jelly for treatment or prevention of dry skin. Most preferably, the active ingredient is a diaper rash ointment for treatment and prevention of diaper rash. A powder treatment for absorption of moisture may be provide as the active ingredient, and may include baby powders, particularly corn starch based powders. The active ingredient segment may be integrated within the portion of the skin-contact surface so that it is interstitially integrated within fibers of the skin-contact surface to provide transfer of the active ingredients. Alternatively, the active ingredient segment is applied as a coating upon the portion of the skin-contact surface to provide transfer of the active ingredients, the coating being comprised of at least one active ingredient layer. The portion of the skin-contact surface with the active ingredient segment integrated therein could be the entire surface thereof, or may simply be a zone or section of the skin-contact surface, such as a soiling zone extending between the crotch area and the buttocks area of an infant being diapered.

In another embodiment, a diaper pad having active ingredients integrated therein is provided. The diaper pad is separate from a diaper and is adapted to be secured within a diaper when the diaper is appointed to be used on an infant. Thus, the diaper pads can be sold in conjunction with a particular diaper package, or can be sold separate from the diaper package. In this manner, one can receive some of the benefits of a diaper with active ingredients integrated therein, as disclosed herein in the aforementioned embodiment, by simply inserting the diaper pad within a conventional diaper. The diaper pad comprises a pad having top layer appointed for contact with an infant's bottom and a bottom layer having an adhesive coating applied thereto that is appointed to be attached to a skin-contact surface of a diaper. An active ingredient segment is provided that is located in association with the top layer of the pad. The active ingredient segment includes active ingredients integrated therein and is adapted for contact and direct delivery to the infant's bottom when the diaper pad is secured into the diaper, and the diaper is placed on and worn by the infant. The active ingredients may be selected from a group consisting of baby oil, petroleum jelly or lotion for treatment or prevention of dry skin. Preferably, the active ingredient is a diaper rash ointment for treatment or prevention of diaper rash. In another embodiment, the active ingredient is a powder treatment for absorption of moisture and the active ingredient segment comprises an inner powder releasing pouch in association with the top layer of the pad.

Another embodiment provides a diaper wipe having active ingredients integrated therein. The diaper wipe comprises a top wipe layer appointed for contact with an infant's bottom, a bottom layer, and an active ingredient segment located in association with at least one portion of the top wipe layer. The active ingredient segment includes active ingredients therein and is adapted for contact and direct delivery of the active ingredients to the infant's bottom when the wipe is wiped against the infant.

### BRIEF DESCRIPTION OF THE DRAWINGS

The invention will be more fully understood and further advantages will become apparent when reference is had to the following detailed description of the preferred embodiments of the invention and the accompanying drawings, in which:

FIG. 1a illustrates a top view of an embodiment of the diaper having active ingredients integrated therein comprising a skin-contact surface appointed for contact with an infant's bottom, at least one inner absorption layer, an outer most layer, and an active ingredient segment, wherein the portion of the skin-contact surface appointed with the active ingredient segment is a soiling zone extending from the front area of the diaper past the buttocks area;

FIG. 1b illustrates an embodiment, via a side cross-sectional view of the diaper having active integrated therein of FIG. 1a taken along line x-x, wherein the active ingredient segment is integrated within the portion of the skin-contact surface;

FIG. 1c illustrates an embodiment, via a side cross-sectional view of the diaper having active integrated therein of FIG. 1a taken along line x-x, wherein the active ingredient segment is an active ingredient coating applied on the top of the portion of the skin-contact surface;

FIG. 2a illustrates a top view of an embodiment of the diaper having active ingredients integrated therein comprising a skin-contact surface appointed for contact with an infant's bottom, at least one inner absorption layer, an outer most layer, and an active ingredient segment, wherein the active ingredient segment is an active ingredient coating applied on the top of the portion of the skin-contact surface (as shown in FIG. 1c) and a removable peel tab is located thereon;

FIG. 2b illustrates an embodiment, via a side cross-sectional view of the diaper having active integrated therein of FIG. 2a taken along line y-y, showing the active ingredient segment coating applied on the top of the portion of the skin-contact surface and the removable peel tab in place, appointed to be removed when the diaper is being donned;

FIG. 3a illustrates a top view of an embodiment of the diaper having active ingredients integrated therein wherein the portion of the skin-contact surface appointed with the active ingredient segment is a soiling zone extending from the front area of the diaper past the buttocks area;

FIG. 3b illustrates a top view of an embodiment of the diaper having active ingredients integrated therein wherein the portion of the skin-contact surface appointed with the active ingredient segment is a buttocks zone extending slightly from the crotch area of the diaper into the buttocks area;

FIG. 4a illustrates a top view of an embodiment of the diaper having active ingredients integrated therein wherein the active ingredients is a powder treatment for absorption of moisture and the active ingredient segment comprises an inner powder releasing pouch in association with the portion of the skin-contact surface located between the skin-contact surface and the inner absorption layer of the diaper;

FIG. 4b illustrates a cross-sectional view of the diaper in FIG. 4a taken along V-V;

FIG. 5a illustrates a top view of an embodiment of the diaper pad having active ingredients integrated therein, showing the diaper pad is placed and adhered to the diaper appointed to be diapered onto an infant;

FIG. 5b illustrates a planer view of the diaper pad having active ingredients integrated therein, showing the diaper pad before same is placed and adhered onto the diaper, as shown in FIG. 5a;

FIG. 6a illustrates a top view of an embodiment of the diaper wipe having active ingredients integrated therein, showing the diaper wipe in a folded condition as when stored or before use; and

FIG. 6b illustrates a top view of the diaper wipe of FIG. 6a wherein the diaper wipe is opened and the active ingredient segment side is exposed and ready to be wiped onto an infant's bottom and distributed thereon.

### DETAILED DESCRIPTION OF THE INVENTION

The present invention provides a diaper, diaper pad insert and/or a diaper wipe with active ingredients integrated therein, including ointments for treating diaper rash, baby oils and lotions for treating dry skin, and/or powder treatments for absorbing moisture, including baby powder, corn starch powders and the like. Direct application of the diaper rash ointment, baby oil, lotion and/or powder is achieved while the diaper is being donned and worn as the active ingredient segment is wiped against the infant's body the active ingredients are transferred onto the infant's skin.

Topical application during diapering requires the caregiver/parent to have to fumble with ointment containers/tubes when diapering an infant, which can be quite a challenge when the infant is very active and not wanting to lay still on a changing table or surface. During travel, such ointments are just one more item that needs to be toted around in the diaper bag and dealing with same when using a restroom changing table can be quite difficult. In fact, the situation can be dangerous, as the caregiver must take both hands off of the infant to open the ointment container, leading to the risk of the infant rolling from the changing surface. Moreover, the topical application results in residual ointment or lotion being deposited on the caregiver's fingers. In a perfect situation the caregiver would be able to immediately wash his/her hands. But in reality, the caregiver does not always have that luxury, as the infant cannot be left on the changing table alone while the caregiver goes to wash his/her hands, and it is very difficult washing one's hands while holding an infant. During travel, a sink may not even be readily available, as parents often must change the infant's/baby's diaper in a vehicle or stroller. Realistically, the ointment cannot be immediately washed from the hands after every application, and is transferred onto clothing, or worse, onto bottles and nipples being prepared for feeding the infant, or onto the breast, ultimately risking ingesting by the infant.

In the embodiments concerning the diaper and diaper pad, during wear, the diaper (and/inserted diaper pad) with active ingredients integrated therein comes into contact with the infant's bottom and transfers the active ingredients thereto. Through long intervals, the infant is protected and the risk of developing (or worsening) diaper rash conditions is mitigated. The diaper should optimally be changed as soon as possible after each soiling as a usual diaper, but the diaper (and/inserted diaper pad) herein facilitates in protecting the infant's body during wear and provides advantages during the diapering process. In the embodiments concerning the diaper wipe, the user simply utilizes the wipes to clean the infant, and when doing so the active ingredients are transferred to the infant's bottom. Preferably, the caregiver or parent utilizes a conventional wipe to clean the soiled infant, and then utilizes the diaper wipe with active ingredients thereon of the subject invention on the infant to distribute the active ingredients thereon right before the diaper is placed onto the infant, as the last, final wipe.

Advantageous include: (i) protecting an infant from diaper rash during prolonged periods where the infant is not changed—such as during the night, extended periods of sleep, naptime, or during travel; (ii) convenience during the diapering process, allowing the caregiver to simply place the diaper on the child without the need to apply separate ointments, lotions, and/or powder, which can be very advantageous when dealing with a squirming toddler; (iii) provides the caregiver the ability to apply the ointment, or other medicament, without using their hands so that their hands do not have residual ointment or lotion on them; and (iv) results in a lighter, more compact diaper bag, as less items are necessary to carry along in the bag, so that the caregiver will not have to tote around lotions, ointments and powders in the bag, instead the caregiver will need to only carry the diaper and/or diaper wipes with active ingredients integrated therein.

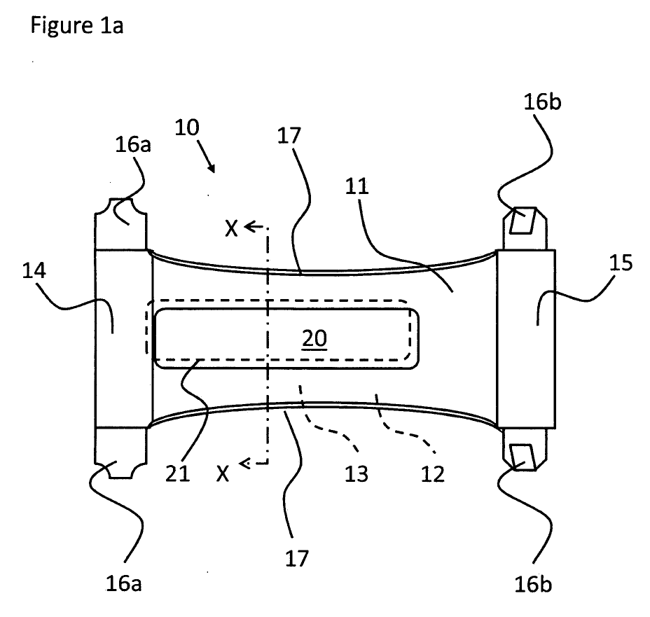

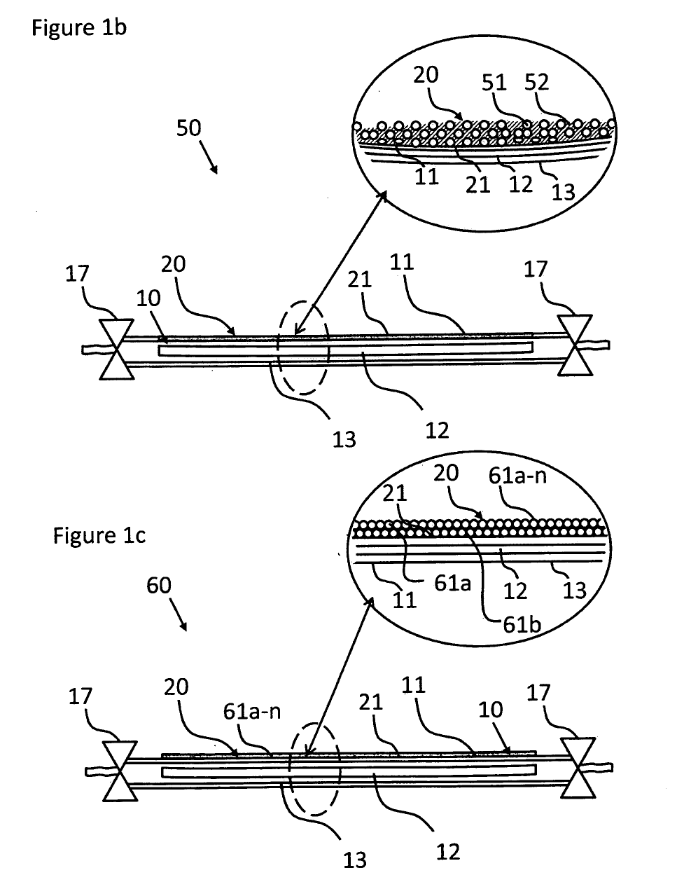

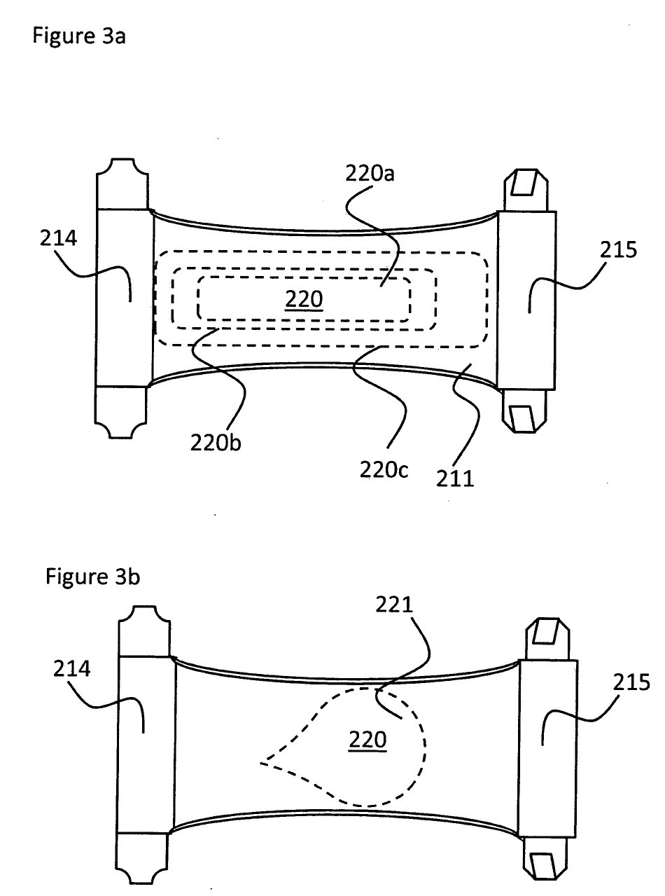

FIG. 1a illustrates a top view of an embodiment of the diaper having active ingredients integrated therein, shown generally at 10. Diaper 10 comprises a skin-contact surface 11 appointed for contact with an infant's bottom, at least one inner absorption layer 12, and an outer most layer 13. Diaper 10 is constructed having a front waist band 14 and a back waist band 15 with mating attachment means 16a, 16b, and leg perimeter zones 17. Diaper 10 further comprises an active ingredient segment 20 located over a portion 21 of skin-contact surface 11, herein portion 21 is shown in a soiling zone extending from an area near the front waist band 14 past the crotch area and into the buttocks area towards the back waist band 15. The portion 21 with the active ingredient segment 20 thereon/therewith may cover the entire surface of the skin contact layer 11. Preferably, portion 21 with active ingredient segment 20 thereon/therewith is a soiling zone extending from a crotch area to a buttocks area of the diaper so that the active ingredient segment provides contact and dissemination of the active ingredients to crotch and buttocks areas of the infant while the diaper is being donned and worn, as is generally shown in this figure. In another embodiment, the portion 21 may simply be provided as a buttocks zone extending over a buttocks area of the diaper so that the active ingredient segment provides contact and dissemination of the active ingredients to the buttocks of the infant while the diaper is being donned and worn. (See FIGS. 3a and 3b).

The active ingredient segment 20 includes active ingredients associated therewith and is adapted for contact and direct delivery to the infant's bottom when the diaper 10 is placed on and worn by the infant. The active ingredient segment 20 is located in association with the skin-contact surface 11 of the diaper 10 so that the active ingredients coated upon or integrated within the skin-contact surface 11 via active ingredient segment 20 comes into contact with the infant's bottom. As the infant wears the diaper and moves around, the skin-contact surface 11, and visa vie the active ingredient segment 20, comes into contact with the infant's body, transferring active ingredients associated with the active ingredient segment 20 onto the infant's body. This active ingredient may be a lotion, baby oil or petroleum jelly for treatment or prevention of dry skin. Most preferably, the active ingredient is a diaper rash ointment for treatment and prevention of diaper rash. A powder treatment for absorption of moisture may be provide as the active ingredient, and may include baby powders, particularly corn starch based powders.

FIGS. 1b and 1c illustrated embodiments of the application of the active ingredient segment 20 as integrated with skin contact layer 11 of the diaper 10 shown in FIG. 1a as taken along line x-x. Particularly, FIG. 1b illustrates an embodiment wherein the active ingredient segment 20 is integrated within the portion 21 of the skin-contact surface 11, shown generally at 50. In this manner, the integration may be achieved during the manufacturing process by various methods, including by spray, or micro-spray application onto the skin contact layer 11 during production of the diaper 10. Particles of the active ingredient 51 thus result so that active ingredient segment 20 is interstitially integrated within fibers 52 of the skin contact layer 11 in the designated portion 21. FIG. 1c illustrates an embodiment wherein the active ingredient segment 20 is provided as an active ingredient coating 61 layers 61a-n applied over the portion 21 of the skin-contact surface 11, shown generally at 60. In this manner, the integration may be achieved during the manufacturing process by various methods, including by roll or spray application onto the skin contact layer 11 during production of the diaper 10, in a manner so as to provide layers 61a, 61b of the active ingredient. Particles of the active ingredient thus result in a series of layers 61a-n that yield a relatively tacky active ingredient segment 20. Preferably, there is at least one coating layer 61a, more preferably there is at least three coating layers 61a-c or a plurality of coating layers 61a-n. The thickness of each individual layer 61a, 61b may vary from one another, or may be equal to one another. Preferably, each individual layer 61a-61n has an individual thickness ranging between 0.025 cm up to 1.27 cm in thickness {each}, preferably ranging from 0.025 cm up to 0.635 cm {each}. Preferably, the overall thickness of the sum of the layers 61a-n combined should optimally not exceed 1.27 cm. In this embodiment, when diapering, optimal coverage can be gained by the caregiver swiping the active ingredient segment 20 against the infant's bottom and then closing the diaper. Thus, initial delivery is achieved when the diaper is being placed on the infant, and continuous delivery is achieved as the diaper is being worn and the active ingredient segment 20 rubs against or comes into contact with the infant's bottom.

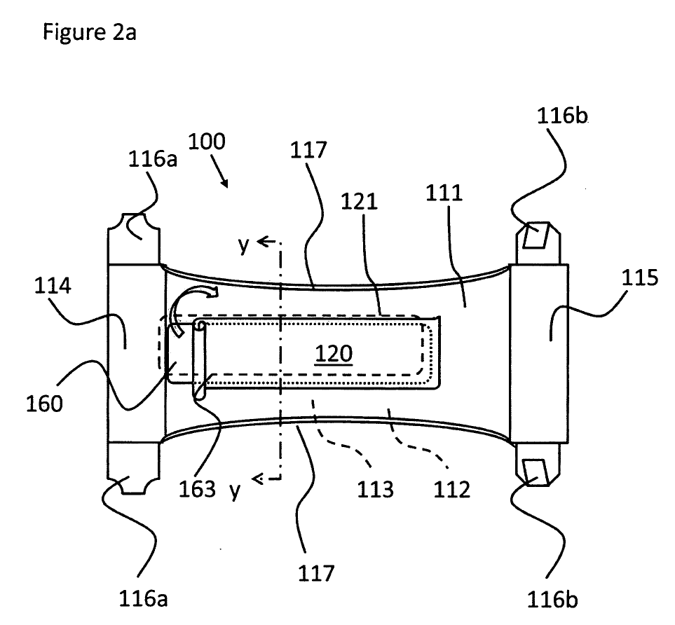

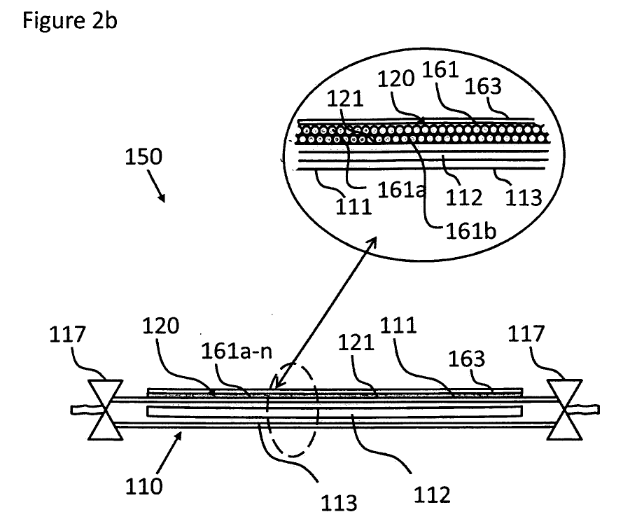

FIG. 2a illustrates a top view of an embodiment of the diaper having active ingredients integrated therein comprising a skin-contact surface appointed for contact with an infant's bottom, at least one inner absorption layer, an outer most layer, and an active ingredient segment, wherein the active ingredient segment is an active ingredient coating applied on the top of the portion of the skin-contact surface (as shown in FIG. 1c) and a removable peel tab is located thereon, shown generally at 100. FIG. 2b illustrates an embodiment, via a side cross-sectional view of the diaper having active integrated therein of FIG. 2a taken along line y-y, showing the active ingredient segment coating applied on the top of the portion of the skin-contact surface and the removable peel tab in place, appointed to be removed when the diaper is being donned, shown generally at 150. Diaper 100 comprises a skin-contact surface 111 appointed for contact with an infant's bottom, at least one inner absorption layer 112, and an outer most layer 113. Diaper 100 is constructed having a front waist band 114 and a back waist band 115 with mating attachment means 116a, 116b, and leg perimeter zones 117. Diaper 100 further comprises an active ingredient segment 120 located over a portion 121 of skin-contact surface 111, herein portion 121. The active ingredient segment 120 includes active ingredients associated therewith and is adapted for contact and direct delivery to the infant's bottom when the diaper 100 is placed on and worn by the infant. The active ingredient segment 120 is located in association with the skin-contact surface 111 of the diaper 100 so that the active ingredients are applied as a coating 161 preferably composed of a plurality of layers. Particles of the active ingredient thus result in a series of layers 161 (161a-n) that yield a relatively tacky active ingredient segment 120. Further provided is a peel tab 163 that covers and protects the active ingredients of segment 120 from drying out or degrading during non-use or in storage. Removably peel tab 163 is generally comprised as a peel tab having a smooth, non-porous surface, or smooth surface with a polymeric coating or wax coating, or the like, that comes into intimate contact with the active ingredient segment 120 and can readily be peeled therefrom; conversely, the top of the peel tab 163 may be a non-coated surface. The peel tab 163 is similar to those utilized when dealing with tacky surfaces or adhesive surfaces. In this embodiment, when diapering, the caregiver simply opens the diaper 100 as usual and aligns the diaper 100 with the infant's bottom and peels back peel tab 163 and removes same to expose the active ingredient segment 120. Optimal coverage can be gained by the caregiver then swiping the active ingredient segment 120 against the infant's bottom and then closing the diaper. Thus, initial delivery is achieved when the diaper is being placed on the infant, and continuous delivery is achieved as the diaper is being worn and the active ingredient segment 120 rubs against or comes into contact with the infant's bottom.

FIG. 3a illustrates a top view of an embodiment of the diaper having active ingredients integrated therein wherein the portion of the skin-contact surface 211 appointed with the active ingredient segment 220 is shown in a series of configurations, shown in phantom 220a0c, generally running from towards the front area (front waist band 214) of the diaper past the buttocks area (towards back waist band 215). FIG. 3b illustrates a top view of an embodiment of the diaper having active ingredients integrated therein wherein the portion of the skin-contact surface 212 appointed with the active ingredient segment is a buttocks zone 221 extending slightly from the crotch area of the diaper into the buttocks area at 250. The portion or zone with the active ingredient segment thereon may be of any 'shape or form, but preferably is provided to align with and be parallel with the infant's buttocks area and at least a portion of the crotch area, as these areas are most prone to a diaper rash breakout.

In operation, the caregiver/parent removes the infant's soiled diaper and cleans the infant with diaper wipes or the like, or gives the infant a bath. After the infant is cleaned, using conventional methods, the caregiver would have to open the diaper ointment, or powder, etc. and topically apply the ointment or powder onto the infant, followed by diapering of the infant. However, in operation of the diaper with active ingredients herein, the caregiver simply opens the diaper to expose the skin-contact surface and slips the diaper under the infant, wherein a peel tab is provided over the active ingredients, the tab is removed, and the caregiver closes the front and back waist bands of the diaper around the infant's hips and fasteners same with fasteners.

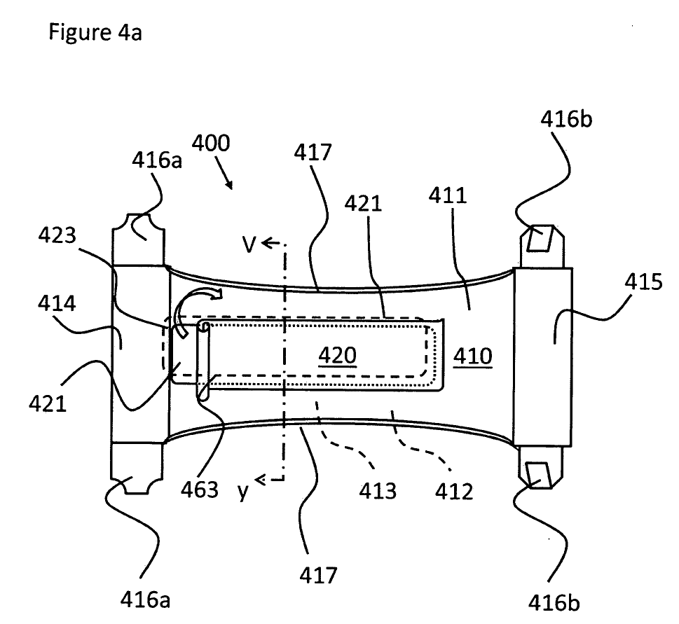

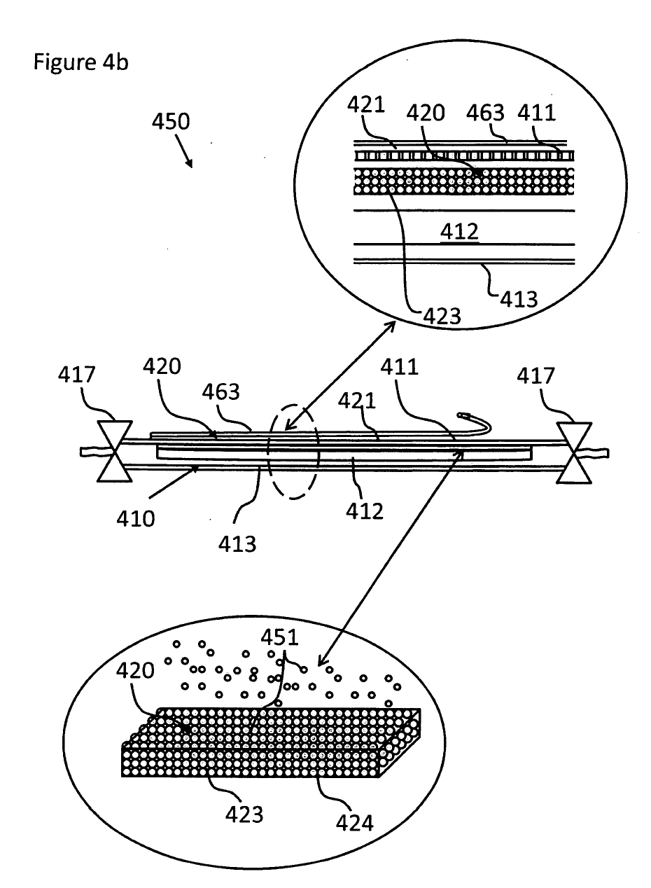

FIG. 4a illustrates a top view of an embodiment of the diaper having active ingredients integrated therein wherein the active ingredients is a powder treatment for absorption of moisture and the active ingredient segment comprises an inner powder releasing pouch, shown generally at 400. Diaper 410 comprises a skin-contact surface 411 appointed for contact with an infant's bottom, at least one inner absorption layer 412, an outer most layer 413, and an active ingredient segment 420 located under a portion 421 of skin contact layer 411. Diaper 410 is constructed having a front waist band 414 and a back waist band 415 with mating attachment means 416a, 416b, and leg perimeter zones 417. The diaper preferably includes a top peel tab 463, which when peeled back exposes portion 421 and opens active ingredient segment 420 under portion 421 of skin-contact surface 411. Skin contact surface 411, or at least portion 421 thereof, is composed of a mesh or knitted material having apertures therein for escape of powder from active ingredient segment 420, which comprises an inner powder releasing pouch 423 housing powdered active ingredients. FIG. 4b illustrates a cross-sectional view of diaper 410 taken along V-V, shown generally at 450. Diaper 410 is provided having active ingredients provided as a powder treatment 451 for absorption of moisture which is released from the active ingredient segment 420 and skin-contact surface 411. Active ingredient segment 420 comprises an inner powder releasing pouch 423 in association with the portion 421 of skin-contact surface 411, located between skin-contact surface 411 and inner absorption layer 412. Powder releasing pouch 423 comprises a mesh housing 424 containing powder 451 therein so that powder 451 is released from mesh housing 424, and visa vie powder releasing pouch 423, when diaper 410 is being worn. As the powder 451 is released from pouch 423, it continues to traverse through holes/a mesh material making up skin-contact surface 411 so that the powder 451 is released to contact the infant's bottom and provide moisture absorption and protection thereto. Preferably, diaper 410 further includes a removable peel tab 463 located over active ingredient segment 420 and portion 421, and visa vie pouch 423, so that the powder 451 does not escape or become exposed to moisture penetration during storage and before use. Preferably the powder 451 is a baby powder composed of a corn starch material.

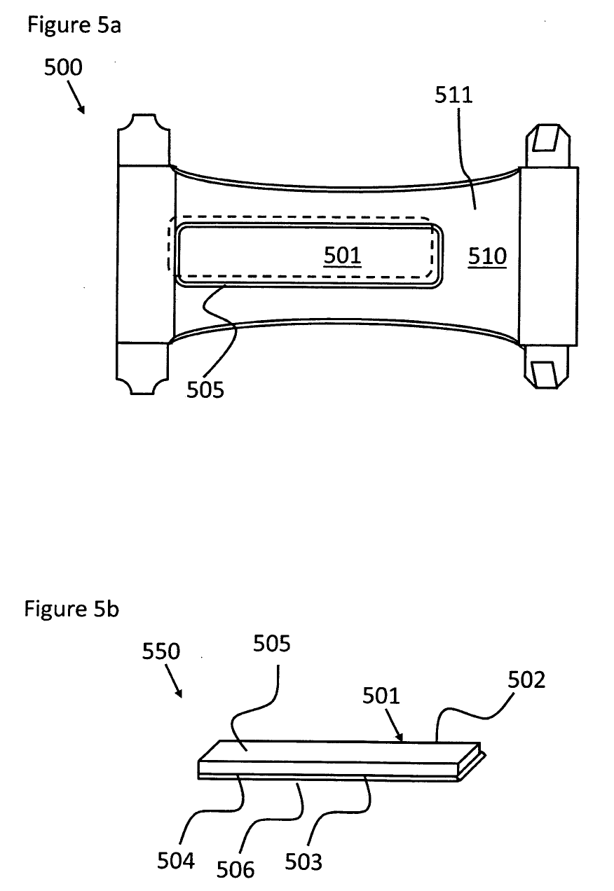

FIG. 5a illustrates a top view of an embodiment of the diaper pad having active ingredients integrated therein, showing the diaper pad is placed and adhered to the diaper appointed to be diapered onto an infant, shown generally at 500. FIG. 5b illustrates a planer view of the diaper pad having active ingredients integrated therein; showing the diaper pad before same is placed and adhered onto the diaper, as shown in FIG. 5a, shown generally at 550. A diaper pad 501 having active ingredients integrated therein is provided. The diaper pad 501 is separate from a diaper 510 and is adapted to be secured within the diaper 510 when the diaper 510 is appointed to be used on an infant. Thus, the diaper pads 501 can be sold in conjunction with a particular diaper package, or can be sold separate from the diaper package. In this manner, one can receive some of the benefits of a diaper with active ingredients integrated therein, as disclosed herein in the aforementioned embodiment, by simply inserting the diaper pad 501 within a conventional diaper 510. The diaper pad 501 comprises a top layer 502 appointed for contact with an infant's bottom and a bottom layer 503 having an adhesive coating 504 applied thereto that is appointed to be attached to a skin-contact surface 511 of diaper 510. Preferably, a pad peel tab 506 is provided over the adhesive coating 504 and is appointed to be peeled from the adhesive coating 504 when the pad 501 is to be used. An active ingredient segment 505 is provided that is located in association with the top layer 502 of the pad 501. The active ingredient segment 505 includes active ingredients integrated therein and is adapted for contact and direct delivery to the infant's bottom when the diaper pad 501 is secured into the diaper, and the diaper is placed on and worn by the infant. The active ingredients may be selected from a group consisting of baby oil, petroleum jelly or lotion for treatment or prevention of dry skin. Preferably, the active ingredient is a diaper rash ointment for treatment or prevention of diaper rash. A top peel tab may also be provided that is appointed to protect the active ingredients integrated or coated on the top layer 502 of the pad 501. In another embodiment, the active ingredient is a powder treatment for absorption of moisture and the active ingredient segment comprises an inner powder releasing pouch in association with the top layer of the pad (similar to the configuration discussed and shown in FIGS. 4a and 4b). Preferably, the active ingredients are applied as a coating on the top layer 502 of the pad 501.

In operation of the diaper pad herein, a user opens a diaper appointed to be placed onto an infant. The user then takes a diaper pad 501 and peels off pad peel tab 506 to expose the adhesive coating 504 located on bottom layer 503. Next, the user aligns diaper pad 501 with the skin contact layer 511 of the diaper 510 and firmly presses the diaper pad 501 onto the skin contact layer 511, causing the adhesive coating 504 to adhere to the skin contact layer 511. Where a top peel tab 507 is provided, the top peel tab 507 is then removed from the pad 501 to expose the active ingredient segment 505. The diaper is then fastened.

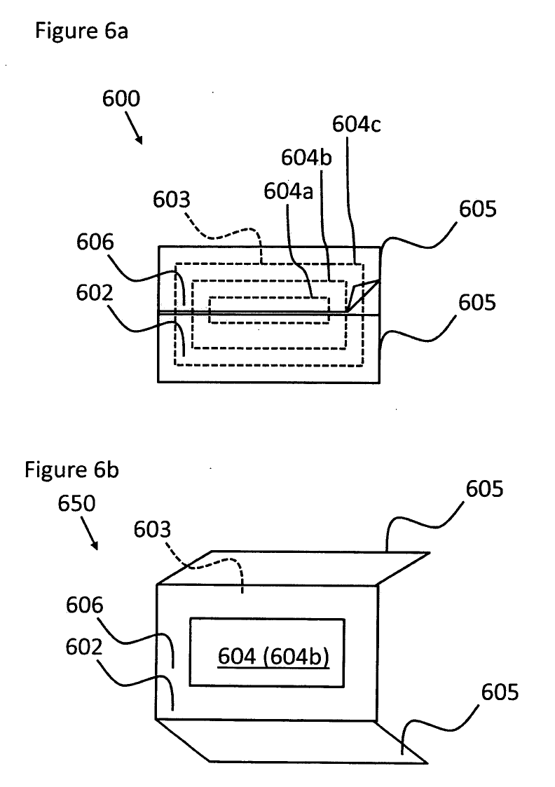

FIG. 6a illustrates a top view of an embodiment of the diaper wipe having active ingredients integrated therein, showing the diaper wipe in a folded condition as when stored or before use, shown generally at. 600. FIG. 6b illustrates a top view of the diaper wipe of FIG. 6a wherein the diaper wipe is opened and the active ingredient segment side is exposed and ready to be wiped onto an infant's bottom and distributed thereon, shown generally at 650. Wipes in generally are provided as a three-fold construction, with two flaps 605 folded over a central portion 606. The diaper wipe comprises the central portion 606 with a top wipe layer 602 appointed for contact with an infant's bottom, a bottom layer 603, and an active ingredient segment 604 located in association with at least one portion of the top wipe layer 602. Herein, active ingredient segments 604 are shown in phantom to represent placement and size of the active ingredient segment 604. Particularly, the active ingredient segment 604 may be a small strip, centrally located shown at 604a on central portion 606; a slightly larger rectangular strip centrally located shown at 604b, a larger strip area shown at 604c; or a large are 604d that basically covers a substantial portion of the top wipe layer 602. In use, top layer 602 is exposed by unfolding flaps 605, which in turn exposes active ingredient segment 604 (as shown in FIG. 6b, 604b for example). The wipe is then arranged so that the active ingredient segment 604 comes into contact with the infant's bottom for contact and direct delivery of the active ingredients to the infant when the wipe is wiped against the infant. In operation, preferably the baby's soiled bottom is first cleaned with conventional wipes, or through other cleaning means. After the infant's bottom is cleaned and ready for diapering, the diaper wipe 601 is utilized to deliver active ingredients to the infant's bottom. The wipe is then discarded and the diaper secured to the infant. In this manner, the caregiver does not have to directly apply the ointment 5 or active ingredients with his/her hands and the hands remain free of residual ointment. The active ingredients are preferably a diaper rash ointment for treatment or prevention of diaper rash. Alternatively, the active ingredients may be selected form a group consisting of baby oil, petroleum jelly or lotion for treatment or prevention of dry skin. Preferably, the active ingredient segment is applied as at least one active ingredient coating over the portion of the top wipe layer.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A diaper having active ingredients integrated therein, comprising:
a. a skin-contact surface appointed for contact with an infant's bottom, at least one inner absorption layer, and an outer most layer; and
b. an active ingredient segment having said active ingredients integrated therein located on said skin-contact surface;
wherein said active ingredient segment is adapted to directly said active ingredients to said infant's bottom when said diaper is placed on and worn by said infant.

2. A diaper having active ingredients integrated therein as recited by claim 1, wherein said active ingredients is a diaper rash ointment for treatment or prevention of diaper rash.

3. A diaper having active ingredients integrated therein as recited by claim 1, wherein said active ingredients is selected from a group consisting of baby oil, petroleum jelly or lotion for treatment or prevention of dry skin.

4. A diaper having active ingredients integrated therein as recited by claim 1, wherein said active ingredients is a powder treatment for absorption of moisture.

5. A diaper having active ingredients integrated therein as recited by claim 1, wherein said portion of said skin-contact surface associated with said active ingredient segment is a soiling zone extending from a crotch area to a buttocks area of said diaper so that said active ingredient segment provides contact and dissemination of said active ingredients to crotch and buttocks areas of said infant while said diaper is being donned and worn.

6. A diaper having active ingredients integrated therein as recited by claim 1, wherein said portion of said skin-contact surface associated with said active ingredient segment is a buttocks zone extending over a buttocks area of said diaper so that said active ingredient segment provides contact and dissemination of said active ingredients to said buttocks of said infant while diaper is being donned and worn.

7. A diaper having active ingredients integrated therein as recited by claim 1, wherein said active ingredient segment is integrated within said portion of said skin-contact surface so that it is interstitially integrated within fibers of said skin-contact surface to provide transfer of said active ingredients.

8. A diaper having active ingredients integrated therein as recited by claim 1, wherein said active ingredient segment is applied as a coating upon said portion of said skin-contact surface to provide transfer of said active ingredients, said coating being comprised of at least one active ingredient layer.

9. A diaper having active ingredients integrated therein as recited by claim 8 comprising a removably peel tab located over said coating of said active ingredients to prevent degradation or drying of said active ingredients during storage and before use.

10. A diaper having active ingredients integrated therein as recited by claim 9, wherein said active ingredients is a diaper rash ointment.

11. A diaper having active ingredients integrated therein as recited by claim 9, wherein said active ingredients is selected from a group consisting of baby oil, petroleum jelly or lotion.

12. A diaper having active ingredients integrated therein as recited by claim 1, wherein said active ingredients is a powder treatment for absorption of moisture and said active ingredient segment comprises an inner powder releasing pouch in association with said portion of said skin-contact surface located between said skin-contact surface and said inner absorption layer of said diaper, said powder releasing insert comprising a mesh pouch housing a powder therein so that said powder is released from said pouch when said diaper is being worn and is released through said pouch and through said skin-contact surface of said diaper and contacts said infant's bottom to provide moisture absorption and protection thereto.

13. A diaper pad having active ingredients integrated therein, comprising:
a. a pad having top layer appointed for contact with an infant's bottom and a bottom layer having an adhesive coating applied thereto being appointed to be adhered to a skin-contact surface of a diaper; and
b. an active ingredient segment having said active ingredients integrated therein located on said top layer of said pad;
wherein said active ingredient segment is adapted to directly delivery said active ingredients to said infant's bottom when said diaper pad is adhered to said skin-contact surface of said diaper and said diaper is placed on and worn by said infant.

14. A diaper pad having active ingredients integrated therein as recited by claim 13, wherein said active ingredients is a diaper rash ointment for treatment or prevention of diaper rash.

15. A diaper pad having active ingredients integrated therein as recited by claim 13, wherein said active ingredients is selected from a group consisting of baby oil, petroleum jelly or lotion for treatment or prevention of dry skin.

16. A diaper pad having active ingredients integrated therein as recited by claim 13, wherein said active ingredients is a powder treatment for absorption of moisture and said active ingredient segment comprises an inner powder releasing pouch in association with said top layer located between said top layer and said bottom layer of said pad, said powder releasing insert comprising a mesh pouch housing a powder therein so that said powder is released from said pouch when said diaper is being worn and is released through said pouch and through said top layer of said pad and contacts said infant's bottom to provide moisture absorption and protection thereto.

17. A diaper pad having active ingredients integrated therein as recited by claim 13, wherein said active ingredient segment is applied as a coating upon said top layer to provide transfer of said active ingredients, said coating being comprised of at least one active ingredient layer.

18. A diaper wipe having active ingredients integrated therein, comprising:
a. a top wipe layer appointed for contact with an infant's bottom and a bottom layer; and
b. an active ingredient segment having said active ingredients integrated therein located on said top wipe layer;
wherein said active ingredient segment is adapted to directly delivery said active ingredients to said infant's bottom when said top wipe layer is wiped against said infant in preparation for diapering said infant.

19. A diaper wipe having a active ingredients integrated therein as recited by claim 18, wherein said active ingredients is a diaper rash ointment for treatment or prevention of diaper rash.

20. A diaper wipe having active ingredients integrated therein as recited by claim 18, wherein said active ingredients is selected form a group consisting of baby oil, petroleum jelly or 20 lotion for treatment or prevention of dry skin.

21. A diaper wipe having active ingredients integrated therein as recited by claim 18, wherein said active ingredients segment is applied as at least one active ingredient coating over said portion of said top wipe layer.
